SEQUENCE LISTING (134 sequences)

SEQ ID NO: 1 - DNA, 1983 bp, Artificial Sequence
  ADF3-Kai_c1e1f Gene 943
atgcaccatcaccatcaccatcatcaccatcattcatcggggtcatctcttgaagtactgtttcagggtcctgctcgggccggcagcgggcagcaaggaccagggcagcaggggccggggcagcaaggtccgggtcaacagggcccctatggcccaggagctagcgctgccgccgctgccgcagggggttatggccctggctccgggcagcagggcccctctcaacagggaccaggtcagcagggtcctgggggtcaagggccttacggccctggagcatccgctgctgccgctgccgctggggggtatggcccaggatccggtcagcaaggacccggcggtcaaggtccatacggtccaggttccagcgctgcagcggcagccgcaggcgggaatgggccaggaagcggtcaacagggcgcggggcaacagggtccgggacaacaaggcccgggcgccagtgcagccgccgccgctgccggcgggtatggtcctgggtctggacagcagggaccaggccaacaaggtcctggagggcaagggccctatggtccgggggcttcggcagcagcagccgccgcaggtgggtacggacctgggtcgggccaaggaccgggccaacaaggcccaggaggtcagggaccgtatggaccaggcgcaagcgcggctgcggcagctgcaggtggatacggtcccggcagcggacagcaagggcctggacaacagggacctggccagcagggaccgggcggtcagggcccttacgggccaggtgcaagtgccgcagcagcagcggctggagggtacggacctggttatggacagcaaggccccggtcagcaggggcctggcggccaagggccttatggaccgggtgctagcgccgcaagtgcagccagtgggggatacgggcctggttcgggtcaacaaggacccggtcaacaggggcctggaggtcagggtccgtatggtccaggtgcatcggcagcagctgcagccgccggtggatacgggcccgggagcggacaacaaggtccgggccagcaaggtccaggacaacaaggccccggccagcaaggtcccggaggccaaggaccttacggaccaggcgcgtctgctgcagcggctgctgcggggggatatggtccaggaagcggccaacaaggacctggccaacaggggccgggtcagcaagggccaggacagcagggcccaggacagcaaggtcctgggcagcagggtcccggacaacaaggaccgggtcaacaaggtcctggtcagcagggaccaggtgggcaaggtgcctatgggcccggtgcctcagcagctgcaggcgcagctgggggctacggtcctggaagtgggcagcaaggcccaggtcagcagggccctggtcaacagggtccgggccaacaagggccaggccaacagggcccagggcaacaagggcccgggcaacaggggccaggtcagcaaggcccttatggtccaggggcatcggccgccgccgcagctgctggtgggtacggcccaggctctggccagcagggccccggacaacagggaccgggacagcagggtccaggaggtcaaggtccttacggtcctggagctgcttccgctgccgtatccgtaggtggctatgggccacaaagtagttctgtcccagtagcctccgcggtggcttcgcgactatcgtcacctgcggcgtcatcgagagttagttcagctgtatcatctcttgtctctagcggtccaactaaacatgcagctttatctaatacgatctcatctgtggtaagccaggtttctgcgtcgaacccaggtttatcaggatgtgatgtattagtccaagcactgttagaggtagtgtctgctcttgtatctatacttggaagttcatcgataggtcaaattaattatggagcttcagcacaatatacacaaatggtaggacaatctgttgcacaagcattagcataa

SEQ ID NO: 2 - DNA, 62 bp, Artificial Sequence
  SEQ ID NO. 1 sense 1-62
atgcaccatcaccatcaccatcatcaccatcattcatcggggtcatctcttgaagtactgtt

SEQ ID NO: 3 - DNA, 62 bp, Artificial Sequence
  SEQ ID NO. 1 antisense 41-102
tggtccttgctgcccgctgccggcccgagcaggaccctgaaacagtacttcaagagatgacc

SEQ ID NO: 4 - DNA, 62 bp, Artificial Sequence
  SEQ ID NO. 1 sense 81-142
cggcagcgggcagcaaggaccagggcagcaggggccggggcagcaaggtccgggtcaacagg

SEQ ID NO: 5 - DNA, 62 bp, Artificial Sequence
  SEQ ID NO. 1 antisense 121-182
gcggcagcggcggcagcgctagctcctgggccataggggccctgttgacccggaccttgctg

SEQ ID NO: 6 - DNA, 62 bp, Artificial Sequence
  SEQ ID NO. 1 sense 161-222
ctagcgctgccgccgctgccgcagggggttatggccctggctccgggcagcagggcccctct

SEQ ID NO: 7 - DNA, 62 bp, Artificial Sequence
  SEQ ID NO. 1 antisense 204-265
aaggcccttgacccccaggaccctgctgacctggtccctgttgagaggggccctgctgcccg

SEQ ID NO: 8 - DNA, 62 bp, Artificial Sequence
  SEQ ID NO. 1 sense 247-308
cctgggggtcaagggccttacggccctggagcatccgctgctgccgctgccgctggggggta

SEQ ID NO: 9 - DNA, 62 bp, Artificial Sequence
  SEQ ID NO. 1 antisense 287-348
accttgaccgccgggtccttgctgaccggatcctgggccataccccccagcggcagcggcag

SEQ ID NO: 10 - DNA, 62 bp, Artificial Sequence
  SEQ ID NO. 1 sense 327-388
gcaaggacccggcggtcaaggtccatacggtccaggttccagcgctgcagcggcagccgcag

SEQ ID NO: 11 - DNA, 57 bp, Artificial Sequence
  SEQ ID NO. 1 antisense 367-423
cgcgccctgttgaccgcttcctggcccattcccgcctgcggctgccgctgcagcgct

SEQ ID NO: 12 - DNA, 47 bp, Artificial Sequence
  SEQ ID NO. 1 sense 402-448
aggaagcggtcaacagggcgcggggcaacagggtccgggacaacaag

SEQ ID NO: 13 - DNA, 52 bp, Artificial Sequence
  SEQ ID NO. 1 antisense 427-478
cagcggcggcggctgcactggcgcccgggccttgttgtcccggaccctgttg

SEQ ID NO: 14 - DNA, 62 bp, Artificial Sequence
  SEQ ID NO. 1 sense 459-520
cagtgcagccgccgccgctgccggcgggtatggtcctgggtctggacagcagggaccaggcc

SEQ ID NO: 15 - DNA, 62 bp, Artificial Sequence
  SEQ ID NO. 1 antisense 499-560
gcccccggaccatagggcccttgccctccaggaccttgttggcctggtccctgctgtccaga

SEQ ID NO: 16 - DNA, 62 bp, Artificial Sequence
  SEQ ID NO. 1 sense 539-600
aagggccctatggtccgggggcttcggcagcagcagccgccgcaggtgggtacggacctggg

SEQ ID NO: 17 - DNA, 62 bp, Artificial Sequence
  SEQ ID NO. 1 antisense 579-640
cctgacctcctgggccttgttggcccggtccttggcccgacccaggtccgtacccacctgcg

SEQ ID NO: 18 - DNA, 62 bp, Artificial Sequence
  SEQ ID NO. 1 sense 621-682
acaaggcccaggaggtcagggaccgtatggaccaggcgcaagcgcggctgcggcagctgcag

SEQ ID NO: 19 - DNA, 62 bp, Artificial Sequence
  SEQ ID NO. 1 antisense 661-722
tgtccaggcccttgctgtccgctgccgggaccgtatccacctgcagctgccgcagccgcgct

SEQ ID NO: 20 - DNA, 55 bp, Artificial Sequence
  SEQ ID NO. 1 sense 701-755
gcggacagcaagggcctggacaacagggacctggccagcagggaccgggcggtca

SEQ ID NO: 21 - DNA, 57 bp, Artificial Sequence
  SEQ ID NO. 1 antisense 734-790
ctgctgcggcacttgcacctggcccgtaagggccctgaccgcccggtccctgctggc

SEQ ID NO: 22 - DNA, 47 bp, Artificial Sequence
  SEQ ID NO. 1 sense 769-815
ccaggtgcaagtgccgcagcagcagcggctggagggtacggacctgg

SEQ ID NO: 23 - DNA, 55 bp, Artificial Sequence
  SEQ ID NO. 1 antisense 794-848
ggcccctgctgaccggggccttgctgtccataaccaggtccgtaccctccagccg

SEQ ID NO: 24 - DNA, 62 bp, Artificial Sequence
  SEQ ID NO. 1 sense 827-888
aaggccccggtcagcaggggcctggcggccaagggccttatggaccgggtgctagcgccgca

SEQ ID NO: 25 - DNA, 62 bp, Artificial Sequence
  SEQ ID NO. 1 antisense 867-928
gttgacccgaaccaggcccgtatcccccactggctgcacttgcggcgctagcacccggtcca

SEQ ID NO: 26 - DNA, 62 bp, Artificial Sequence
  SEQ ID NO. 1 sense 910-971
gggcctggttcgggtcaacaaggacccggtcaacaggggcctggaggtcagggtccgtatgg

SEQ ID NO: 27 - DNA, 62 bp, Artificial Sequence
  SEQ ID NO. 1 antisense 953-1014
cccgtatccaccggcggctgcagctgctgccgatgcacctggaccatacggaccctgacctc

SEQ ID NO: 28 - DNA, 60 bp, Artificial Sequence
  SEQ ID NO. 1 sense 993-1052
tgcagccgccggtggatacgggcccgggagcggacaacaaggtccgggccagcaaggtcc

SEQ ID NO: 29 - DNA, 57 bp, Artificial Sequence
  SEQ ID NO. 1 antisense 1034-1090
ggcctccgggaccttgctggccggggccttgttgtcctggaccttgctggcccggac

SEQ ID NO: 30 - DNA, 61 bp, Artificial Sequence
  SEQ ID NO. 1 sense 1069-1129
ggccagcaaggtcccggaggccaaggaccttacggaccaggcgcgtctgctgcagcggctg

SEQ ID NO: 31 - DNA, 47 bp, Artificial Sequence
  SEQ ID NO. 1 antisense 1108-1154
cttcctggaccatatccccccgcagcagccgctgcagcagacgcgcc

SEQ ID NO: 32 - DNA, 55 bp, Artificial Sequence
  SEQ ID NO. 1 sense 1133-1187
cggggggatatggtccaggaagcggccaacaaggacctggccaacaggggccggg

SEQ ID NO: 33 - DNA, 62 bp, Artificial Sequence
  SEQ ID NO. 1 antisense 1168-1229
ggaccttgctgtcctgggccctgctgtcctggcccttgctgacccggcccctgttggccagg

SEQ ID NO: 34 - DNA, 62 bp, Artificial Sequence
  SEQ ID NO. 1 sense 1210-1271
ggcccaggacagcaaggtcctgggcagcagggtcccggacaacaaggaccgggtcaacaagg

SEQ ID NO: 35 - DNA, 61 bp, Artificial Sequence
  SEQ ID NO. 1 antisense 1252-1312
gcccataggcaccttgcccacctggtccctgctgaccaggaccttgttgacccggtccttg

SEQ ID NO: 36 - DNA, 62 bp, Artificial Sequence
  SEQ ID NO. 1 sense 1294-1355
gggcaaggtgcctatgggcccggtgcctcagcagctgcaggcgcagctgggggctacggtcc

SEQ ID NO: 37 - DNA, 62 bp, Artificial Sequence
  SEQ ID NO. 1 antisense 1336-1397
tgaccagggccctgctgacctgggccttgctgcccacttccaggaccgtagcccccagctgc

SEQ ID NO: 38 - DNA, 62 bp, Artificial Sequence
  SEQ ID NO. 1 sense 1377-1438
aggtcagcagggccctggtcaacagggtccgggccaacaagggccaggccaacagggcccag

SEQ ID NO: 39 - DNA, 47 bp, Artificial Sequence
  SEQ ID NO. 1 antisense 1419-1465
gcccctgttgcccgggcccttgttgccctgggccctgttggcctggc

SEQ ID NO: 40 - DNA, 62 bp, Artificial Sequence
  SEQ ID NO. 1 sense 1444-1505
caagggcccgggcaacaggggccaggtcagcaaggcccttatggtccaggggcatcggccgc

SEQ ID NO: 41 - DNA, 58 bp, Artificial Sequence
  SEQ ID NO. 1 antisense 1484-1541
ccagagcctgggccgtacccaccagcagctgcggcggcggccgatgcccctggaccat

SEQ ID NO: 42 - DNA, 60 bp, Artificial Sequence
  SEQ ID NO. 1 sense 1522-1581
gggtacggcccaggctctggccagcagggccccggacaacagggaccgggacagcagggt

SEQ ID NO: 43 - DNA, 56 bp, Artificial Sequence
  SEQ ID NO. 1 antisense 1564-1619
gaagcagctccaggaccgtaaggaccttgacctcctggaccctgctgtcccggtcc

SEQ ID NO: 44 - DNA, 62 bp, Artificial Sequence
  SEQ ID NO. 1 sense 1600-1661
tacggtcctggagctgcttccgctgccgtatccgtaggtggctatgggccacaaagtagttc

SEQ ID NO: 45 - DNA, 62 bp, Artificial Sequence
  SEQ ID NO. 1 antisense 1642-1703
ggtgacgatagtcgcgaagccaccgcggaggctactgggacagaactactttgtggcccata

SEQ ID NO: 46 - DNA, 57 bp, Artificial Sequence
  SEQ ID NO. 1 sense 1682-1738
tggcttcgcgactatcgtcacctgcggcgtcatcgagagttagttcagctgtatcat

SEQ ID NO: 47 - DNA, 55 bp, Artificial Sequence
  SEQ ID NO. 1 antisense 1717-1771
ctgcatgtttagttggaccgctagagacaagagatgatacagctgaactaactct

SEQ ID NO: 48 - DNA, 62 bp, Artificial Sequence
  SEQ ID NO. 1 sense 1752-1813
cggtccaactaaacatgcagctttatctaatacgatctcatctgtggtaagccaggtttctg

SEQ ID NO: 49 - DNA, 56 bp, Artificial Sequence
  SEQ ID NO. 1 antisense 1792-1847
aatacatcacatcctgataaacctgggttcgacgcagaaacctggcttaccacaga

SEQ ID NO: 50 - DNA, 62 bp, Artificial Sequence
  SEQ ID NO. 1 sense 1826-1887
gtttatcaggatgtgatgtattagtccaagcactgttagaggtagtgtctgctcttgtatct

SEQ ID NO: 51 - DNA, 62 bp, Artificial Sequence
  SEQ ID NO. 1 antisense 1866-1927
ctccataattaatttgacctatcgatgaacttccaagtatagatacaagagcagacactacc

SEQ ID NO: 52 - DNA, 62 bp, Artificial Sequence
  SEQ ID NO. 1 sense 1910-1971
gtcaaattaattatggagcttcagcacaatatacacaaatggtaggacaatctgttgcacaa

SEQ ID NO: 53 - DNA, 31 bp, Artificial Sequence
  SEQ ID NO. 1 antisense 1953-1983
ttatgctaatgcttgtgcaacagattgtcct

SEQ ID NO: 54 - DNA, 3162 bp, Artificial Sequence
  C118
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 55 - DNA, 60 bp, Artificial Sequence
  SEQ ID NO. 54 sense 1-60
agcgcccaatacgcaaaccgcctctccccgcgcgttggccgattcattaatgcagctggc

SEQ ID NO: 56 - DNA, 60 bp, Artificial Sequence
  SEQ ID NO. 54 antisense 41-100
ttgcgctcactgcccgctttccagtcgggaaacctgtcgtgccagctgcattaatgaatc

SEQ ID NO: 57 - DNA, 60 bp, Artificial Sequence
  SEQ ID NO. 54 sense 83-142
agcgggcagtgagcgcaacgcaattaatgtgagttagctcactcattaggcaccccaggc

SEQ ID NO: 58 - DNA, 60 bp, Artificial Sequence
  SEQ ID NO. 54 antisense 125-184
acaattccacacaacatacgagccggaagcataaagtgtaaagcctggggtgcctaatga

SEQ ID NO: 59 - DNA, 60 bp, Artificial Sequence
  SEQ ID NO. 54 sense 163-222
ctcgtatgttgtgtggaattgtgagcggataacaatttcacacaggaaacagctatgacc

SEQ ID NO: 60 - DNA, 60 bp, Artificial Sequence
  SEQ ID NO. 54 antisense 201-260
agaggatccccgggtaccgagctcgaattcgtaatcatggtcatagctgtttcctgtgtg

SEQ ID NO: 61 - DNA, 60 bp, Artificial Sequence
  SEQ ID NO. 54 sense 243-302
ggtacccggggatcctctagagtcgacctgcaggcatgcaagcttggcactggccgtcgt

SEQ ID NO: 62 - DNA, 60 bp, Artificial Sequence
  SEQ ID NO. 54 antisense 285-344
agttgggtaacgccagggttttcccagtcacgacgttgtaaaacgacggccagtgccaag

SEQ ID NO: 63 - DNA, 59 bp, Artificial Sequence
  SEQ ID NO. 54 sense 327-385
ccctggcgttacccaacttaatcgccttgcagcacatccccctttcgccagctggcgta

SEQ ID NO: 64 - DNA, 60 bp, Artificial Sequence
  SEQ ID NO. 54 antisense 368-427
gcaactgttgggaagggcgatcggtgcgggcctcttcgctattacgccagctggcgaaag

SEQ ID NO: 65 - DNA, 60 bp, Artificial Sequence
  SEQ ID NO. 54 sense 407-466
atcgcccttcccaacagttgcgcagcctgaatggcgaatggcgcctgatgcggtattttc

SEQ ID NO: 66 - DNA, 60 bp, Artificial Sequence
  SEQ ID NO. 54 antisense 449-508
tttgacgtatgcggtgtgaaataccgcacagatgcgtaaggagaaaataccgcatcaggc

SEQ ID NO: 67 - DNA, 57 bp, Artificial Sequence
  SEQ ID NO. 54 sense 490-546
tcacaccgcatacgtcaaagcaaccatagtacgcgccctgtagcggcgcattaagcg

SEQ ID NO: 68 - DNA, 60 bp, Artificial Sequence
  SEQ ID NO. 54 antisense 529-588
caagtgtagcggtcacgctgcgcgtaaccaccacacccgccgcgcttaatgcgccgctac

SEQ ID NO: 69 - DNA, 60 bp, Artificial Sequence
  SEQ ID NO. 54 sense 571-630
gcgtgaccgctacacttgccagcgccctagcgcccgctcctttcgctttcttcccttcct

SEQ ID NO: 70 - DNA, 60 bp, Artificial Sequence
  SEQ ID NO. 54 antisense 612-671
atttagagcttgacggggaaagccggcgaacgtggcgagaaaggaagggaagaaagcgaa

SEQ ID NO: 71 - DNA, 58 bp, Artificial Sequence
  SEQ ID NO. 54 sense 651-708
tttccccgtcaagctctaaatcgggggctccctttagggttccgatttagtgctttac

SEQ ID NO: 72 - DNA, 60 bp, Artificial Sequence
  SEQ ID NO. 54 antisense 687-746
accatcacccaaatcaagttttttggggtcgaggtgccgtaaagcactaaatcggaaccc

SEQ ID NO: 73 - DNA, 60 bp, Artificial Sequence
  SEQ ID NO. 54 sense 725-784
aaaacttgatttgggtgatggttcacgtagtgggccatcgccctgatagacggtttttcg

SEQ ID NO: 74 - DNA, 58 bp, Artificial Sequence
  SEQ ID NO. 54 antisense 765-822
gtccactattaaagaacgtggactccaacgtcaaagggcgaaaaaccgtctatcaggg

SEQ ID NO: 75 - DNA, 60 bp, Artificial Sequence
  SEQ ID NO. 54 sense 801-860
tccacgttctttaatagtggactcttgttccaaactggaacaacactcaaccctatctcg

SEQ ID NO: 76 - DNA, 60 bp, Artificial Sequence
  SEQ ID NO. 54 antisense 839-898
gaaatcggcaaaatcccttataaatcaaaagaatagcccgagatagggttgagtgttgtt

SEQ ID NO: 77 - DNA, 60 bp, Artificial Sequence
  SEQ ID NO. 54 sense 877-936
tataagggattttgccgatttcggcctattggttaaaaaatgagctgatttaacaaaaat

SEQ ID NO: 78 - DNA, 60 bp, Artificial Sequence
  SEQ ID NO. 54 antisense 915-974
aattgtaaacgttaatattttgttaaaattcgcgttaaatttttgttaaatcagctcatt

SEQ ID NO: 79 - DNA, 58 bp, Artificial Sequence
  SEQ ID NO. 54 sense 953-1010
caaaatattaacgtttacaattttatggtgcactctcagtacaatctgctctgatgcc

SEQ ID NO: 80 - DNA, 58 bp, Artificial Sequence
  SEQ ID NO. 54 antisense 989-1046
gggtgttggcgggtgtcggggctggcttaactatgcggcatcagagcagattgtactg

SEQ ID NO: 81 - DNA, 60 bp, Artificial Sequence
  SEQ ID NO. 54 sense 1029-1088
cgacacccgccaacacccgctgacgcgccctgacgggcttgtctgctcccggcatccgct

SEQ ID NO: 82 - DNA, 60 bp, Artificial Sequence
  SEQ ID NO. 54 antisense 1071-1130
ctgacacatgcagctcccggagacggtcacagcttgtctgtaagcggatgccgggagcag

SEQ ID NO: 83 - DNA, 59 bp, Artificial Sequence
  SEQ ID NO. 54 sense 1113-1171
gggagctgcatgtgtcagaggttttcaccgtcatcaccgaaacgcgcgagacgaaaggg

SEQ ID NO: 84 - DNA, 60 bp, Artificial Sequence
  SEQ ID NO. 54 antisense 1154-1213
attatcatgacattaacctataaaaataggcgtatcacgaggccctttcgtctcgcgcgt

SEQ ID NO: 85 - DNA, 60 bp, Artificial Sequence
  SEQ ID NO. 54 sense 1192-1251
tataggttaatgtcatgataataatggtttcttagacgtcaggtggcacttttcggggaa

SEQ ID NO: 86 - DNA, 60 bp, Artificial Sequence
  SEQ ID NO. 54 antisense 1234-1293
aatgtatttagaaaaataaacaaataggggttccgcgcacatttccccgaaaagtgccac

SEQ ID NO: 87 - DNA, 60 bp, Artificial Sequence
  SEQ ID NO. 54 sense 1272-1331
tgtttatttttctaaatacattcaaatatgtatccgctcatgagacaataaccctgataa

SEQ ID NO: 88 - DNA, 60 bp, Artificial Sequence
  SEQ ID NO. 54 antisense 1310-1369
atactcatactcttcctttttcaatattattgaagcatttatcagggttattgtctcatg

SEQ ID NO: 89 - DNA, 59 bp, Artificial Sequence
  SEQ ID NO. 54 sense 1348-1406
gaaaaaggaagagtatgagtattcaacatttccgtgtcgcccttattcccttttttgcg

SEQ ID NO: 90 - DNA, 60 bp, Artificial Sequence
  SEQ ID NO. 54 antisense 1387-1446
ccagcgtttctgggtgagcaaaaacaggaaggcaaaatgccgcaaaaaagggaataaggg

SEQ ID NO: 91 - DNA, 60 bp, Artificial Sequence
  SEQ ID NO. 54 sense 1429-1488
ctcacccagaaacgctggtgaaagtaaaagatgctgaagatcagttgggtgcacgagtgg

SEQ ID NO: 92 - DNA, 60 bp, Artificial Sequence
  SEQ ID NO. 54 antisense 1471-1530
tctcaaggatcttaccgctgttgagatccagttcgatgtaacccactcgtgcacccaact

SEQ ID NO: 93 - DNA, 60 bp, Artificial Sequence
  SEQ ID NO. 54 sense 1510-1569
acagcggtaagatccttgagagttttcgccccgaagaacgttttccaatgatgagcactt

SEQ ID NO: 94 - DNA, 60 bp, Artificial Sequence
  SEQ ID NO. 54 antisense 1548-1607
aatacgggataataccgcgccacatagcagaactttaaaagtgctcatcattggaaaacg

SEQ ID NO: 95 - DNA, 60 bp, Artificial Sequence
  SEQ ID NO. 54 sense 1586-1645
tggcgcggtattatcccgtattgacgccgggcaagagcaactcggtcgccgcatacacta

SEQ ID NO: 96 - DNA, 60 bp, Artificial Sequence
  SEQ ID NO. 54 antisense 1628-1687
ttttctgtgactggtgagtactcaaccaagtcattctgagaatagtgtatgcggcgaccg

SEQ ID NO: 97 - DNA, 60 bp, Artificial Sequence
  SEQ ID NO. 54 sense 1666-1725
agtactcaccagtcacagaaaagcatcttacggatggcatgacagtaagagaattatgca

SEQ ID NO: 98 - DNA, 60 bp, Artificial Sequence
  SEQ ID NO. 54 antisense 1704-1763
gttggccgcagtgttatcactcatggttatggcagcactgcataattctcttactgtcat

SEQ ID NO: 99 - DNA, 60 bp, Artificial Sequence
  SEQ ID NO. 54 sense 1746-1805
gataacactgcggccaacttacttctgacaacgatcggaggaccgaaggagctaaccgct

SEQ ID NO: 100 - DNA, 60 bp, Artificial Sequence
  SEQ ID NO. 54 antisense 1788-1847
acgatcaaggcgagttacatgatcccccatgttgtgcaaaaaagcggttagctccttcgg

SEQ ID NO: 101 - DNA, 59 bp, Artificial Sequence
  SEQ ID NO. 54 sense 1827-1885
catgtaactcgccttgatcgttgggaaccggagctgaatgaagccataccaaacgacga

SEQ ID NO: 102 - DNA, 60 bp, Artificial Sequence
  SEQ ID NO. 54 antisense 1867-1926
gcaacgttgttgccattgctacaggcatcgtggtgtcacgctcgtcgtttggtatggctt

SEQ ID NO: 103 - DNA, 57 bp, Artificial Sequence
  SEQ ID NO. 54 sense 1909-1965
caatggcaacaacgttgcgcaaactattaactggcgaactacttactctagcttccc

SEQ ID NO: 104 - DNA, 60 bp, Artificial Sequence
  SEQ ID NO. 54 antisense 1944-2003
aactttatccgcctccatccagtctattaattgttgccgggaagctagagtaagtagttc

SEQ ID NO: 105 - DNA, 60 bp, Artificial Sequence
  SEQ ID NO. 54 sense 1944-2003
ggatggaggcggataaagttgcaggaccacttctgcgctcggcccttccggctggctggt

SEQ ID NO: 106 - DNA, 60 bp, Artificial Sequence
  SEQ ID NO. 54 antisense 2022-2081
agacccacgctcaccggctccagatttatcagcaataaaccagccagccggaagggccga

SEQ ID NO: 107 - DNA, 60 bp, Artificial Sequence
  SEQ ID NO. 54 sense 2063-2122
agccggtgagcgtgggtctcgcggtatcattgcagcactggggccagatggtaagccctc

SEQ ID NO: 108 - DNA, 60 bp, Artificial Sequence
  SEQ ID NO. 54 antisense 2105-2164
atagttgcctgactccccgtcgtgtagataactacgatacgggagggcttaccatctggc

SEQ ID NO: 109 - DNA, 60 bp, Artificial Sequence
  SEQ ID NO. 54 sense 2145-2204
acggggagtcaggcaactatggatgaacgaaatagacagatcgctgagataggtgcctca

SEQ ID NO: 110 - DNA, 60 bp, Artificial Sequence
  SEQ ID NO. 54 antisense 2186-2245
tatgagtaaacttggtctgacagttaccaatgcttaatcagtgaggcacctatctcagcg

SEQ ID NO: 111 - DNA, 60 bp, Artificial Sequence
  SEQ ID NO. 54 sense 2224-2283
tgtcagaccaagtttactcatatatactttagattgatttaaaacttcatttttaattta

SEQ ID NO: 112 - DNA, 60 bp, Artificial Sequence
  SEQ ID NO. 54 antisense 2262-2321
catgagattatcaaaaaggatcttcacctagatccttttaaattaaaaatgaagttttaa

SEQ ID NO: 113 - DNA, 60 bp, Artificial Sequence
  SEQ ID NO. 54 sense 2301-2360
atcctttttgataatctcatgaccaaaatcccttaacgtgagttttcgttccactgagcg

SEQ ID NO: 114 - DNA, 59 bp, Artificial Sequence
  SEQ ID NO. 54 antisense 2343-2401
ggatctcaagaagatcctttgatcttttctacggggtctgacgctcagtggaacgaaaa

SEQ ID NO: 115 - DNA, 60 bp, Artificial Sequence
  SEQ ID NO. 54 sense 2380-2439
tcaaaggatcttcttgagatcctttttttctgcgcgtaatctgctgcttgcaaacaaaaa

SEQ ID NO: 116 - DNA, 60 bp, Artificial Sequence
  SEQ ID NO. 54 antisense 2421-2480
ctcttgatccggcaaacaaaccaccgctggtagcggtggtttttttgtttgcaagcagca

SEQ ID NO: 117 - DNA, 60 bp, Artificial Sequence
  SEQ ID NO. 54 sense 2463-2522
tgtttgccggatcaagagctaccaactctttttccgaaggtaactggcttcagcagagcg

SEQ ID NO: 118 - DNA, 58 bp, Artificial Sequence
  SEQ ID NO. 54 antisense 2505-2562
ctaactacggctacactagaagaacagtatttggtatctgcgctctgctgaagccagt

SEQ ID NO: 119 - DNA, 60 bp, Artificial Sequence
  SEQ ID NO. 54 sense 2541-2600
cttctagtgtagccgtagttaggccaccacttcaagaactctgtagcaccgcctacatac

SEQ ID NO: 120 - DNA, 60 bp, Artificial Sequence
  SEQ ID NO. 54 antisense 2579-2638
ctggcagcagccactggtaacaggattagcagagcgaggtatgtaggcggtgctacagag

SEQ ID NO: 121 - DNA, 60 bp, Artificial Sequence
  SEQ ID NO. 54 sense 2620-2679
taccagtggctgctgccagtggcgataagtcgtgtcttaccgggttggactcaagacgat

SEQ ID NO: 122 - DNA, 60 bp, Artificial Sequence
  SEQ ID NO. 54 antisense 2661-2720
accccccgttcagcccgaccgctgcgccttatccggtaactatcgtcttgagtccaaccc

SEQ ID NO: 123 - DNA, 60 bp, Artificial Sequence
  SEQ ID NO. 54 sense 2700-2759
cggtcgggctgaacggggggttcgtgcacacagcccagcttggagcgaacgacctacacc

SEQ ID NO: 124 - DNA, 60 bp, Artificial Sequence
  SEQ ID NO. 54 antisense 2741-2800
gtggcgctttctcatagctcacgctgtaggtatctcagttcggtgtaggtcgttcgctcc

SEQ ID NO: 125 - DNA, 60 bp, Artificial Sequence
  SEQ ID NO. 54 sense 2781-2840
gagctatgagaaagcgccacgcttcccgaagggagaaaggcggacaggtatccggtaagc

SEQ ID NO: 126 - DNA, 60 bp, Artificial Sequence
  SEQ ID NO. 54 antisense 2822-2881
cctggaagctccctcgtgcgctctcctgttccgaccctgccgcttaccggatacctgtcc

SEQ ID NO: 127 - DNA, 60 bp, Artificial Sequence
  SEQ ID NO. 54 sense 2864-2923
cacgagggagcttccagggggaaacgcctggtatctttatagtcctgtcgggtttcgcca

SEQ ID NO: 128 - DNA, 58 bp, Artificial Sequence
  SEQ ID NO. 54 antisense 2906-2963
ccctgacgagcatcacaaaaatcgacgctcaagtcagaggtggcgaaacccgacagga

SEQ ID NO: 129 - DNA, 60 bp, Artificial Sequence
  SEQ ID NO. 54 sense 2946-3005
ttgtgatgctcgtcaggggggcggagcctatggaaaaacgccagcaacgcggccttttta

SEQ ID NO: 130 - DNA, 60 bp, Artificial Sequence
  SEQ ID NO. 54 antisense 2988-3047
aaagaacatgtgagcaaaaggccagcaaaaggccaggaaccgtaaaaaggccgcgttgct

SEQ ID NO: 131 - DNA, 60 bp, Artificial Sequence
  SEQ ID NO. 54 sense 3027-3086
ccttttgctcacatgttctttcctgcgttatcccctgattctgtggataaccgtattacc

SEQ ID NO: 132 - DNA, 58 bp, Artificial Sequence
  SEQ ID NO. 54 antisense 3065-3122
tcggctgcggcgagcggtatcagctcactcaaaggcggtaatacggttatccacagaa

SEQ ID NO: 133 - DNA, 60 bp, Artificial Sequence
  SEQ ID NO. 54 sense 3101-3160
tgataccgctcgccgcagccgaacgaccgagcgcagcgagtcagtgagcgaggaagcgga

SEQ ID NO: 134 - DNA, 20 bp, Artificial Sequence
  SEQ ID NO. 54 antisense 3143-3163
cttccgcttcctcgctcact

(19) United States
(12) Patent Application Publication
(10) Pub. No.: US 2020/0248231 A1
(43) Pub. Date: Aug. 6, 2020

(54) Method for Producing Double-Stranded DNA Fragments

(71) Applicant: Spiber Inc., Yamagata (JP)

(72) Inventors: Yusuke Ito, Tsuruoka-shi, Yamagata (JP); Chikako Sato, Tsuruoka-shi, Yamagata (JP); Nozomu Yachie, Bunkyo-ku, Tokyo (JP)

(73) Assignee: Spiber Inc., Yamagata (JP)

(21) Appl. No.: 16/641,946

(22) PCT Filed: Aug. 28, 2018
(86) PCT No.: PCT/JP2018/031745

§ 371 Date: Feb. 25, 2020

(30) Foreign Application Priority Data
Aug. 28, 2017 (JP) 2017-163168

(51) Int. Cl. C12Q 1/686

(52) U.S. Cl. CPC C12Q 1/686; C12Q 2531/113

(57) ABSTRACT

The present invention relates to a method for producing a double-stranded DNA fragment having a desired nucleotide sequence through dual asymmetric PCR (DA-PCR). The method comprises: (1) providing a plurality of oligonucleotides (sense oligonucleotides) each corresponding to a part of a sense strand of the double-stranded DNA fragment and a plurality of oligonucleotides (antisense oligonucleotides) each corresponding to a part of an antisense strand of the double-stranded DNA fragment and mixing together the oligonucleotides with equal concentrations, DNA polymerase, and dNTP to prepare a reaction mixture solution; (2) performing PCR by using the reaction mixture solution from step (1); (3) adding a primer set capable of amplifying the double-stranded DNA fragment of full length to the reaction mixture solution from step (2); and (4) performing PCR by using the reaction mixture solution from step (3).

### TECHNICAL FIELD

The present invention relates to a method for producing a double-stranded DNA fragment, in particular, to a method for producing a double-stranded DNA fragment through a DA-PCR method.

### BACKGROUND ART

Polymerase chain reaction (PCR) methods are known as methods of selectively amplifying DNA, which plays a role in succession, expression, and so on of genetic information. PCR requires target DNA as a template; however, target DNA is not always available. In order to efficiently express target DNA in different organisms, it is needed to incorporate a restriction enzyme site or carry out codon optimization for a host cell system.

Used as an alternative method to them is a method of assembling natural or artificial DNA sequences to synthesize the whole gene. However, whole-gene synthesis requires oligonucleotides phosphorylated and purified with polyacrylamide gel for best results, thus being highly expensive.

Known as a method with low cost is a method in which a full-length molecule is prepared through overlap extension PCR (OE-PCR) from oligonucleotides each being 40 nt in length, with their 20 nt overlapped, and covering the full sequences of both strands of a full-length molecule of interest, and the full-length molecule is amplified through PCR with two outer primers (Non Patent Literature 1). It has been reported that by using an improved method, which includes a ligation step before the OE-PCR step of the mentioned method, the full-length DNA of a φX174 bacteriophage was synthesized in 14 days (Non Patent Literature 2).

However, this method suffers from high tendency to cause DNA sequence errors because of the dependence on oligonucleotides, and is not applicable to all genes, although full-length DNA was successfully obtained with the method because φX174 itself was available as an efficient mutation screening tool.

A gene synthesis method with combination of dual asymmetrical PCR (DA-PCR) and OE-PCR has been reported (Non Patent Literature 3), which can be easily automated, and is simple and reproducible with fewer errors and low cost. This method is characterized in that each four continuously aligned oligonucleotides are mixed together to perform DA-PCR in such a manner that the amount of moles of each of the two outer oligonucleotides is excessive and five times that of each of the inner oligonucleotides.

### CITATION LIST

### Non Patent Literature

Non Patent Literature 1: Stemmer W P et al., Gene. 1995 Oct. 16; 164(1):49-53.
Non Patent Literature 2: Hamilton O. Smith et al., Proc. Natl Acad. Sci. MSA, Dec. 23, 2003, Vol. 100, 15440-15445
Non Patent Literature 3: Lei Young et al., Nucleic Acids Research, 2004, Vol. 32, No. 7 e59

### SUMMARY OF INVENTION

### Technical Problem

In particular, in view of the disadvantage of conventional DA-PCR, as a method of amplifying a double-stranded DNA fragment having a desired nucleotide sequence, that only four continuously aligned oligonucleotides are available in one operation, an object of the present invention is to provide an improved DA-PCR method that enables amplification of a double-stranded DNA fragment having a desired nucleotide sequence through DA-PCR with more consecutive oligonucleotides, thus enabling accurate and efficient synthesis of a double-stranded DNA fragment with low cost.

### Solution to Problem

The present inventors found that two-stage dual asymmetric PCR enables accurate and efficient synthesis of DNA, the two-stage dual asymmetric PCR including a step of mixing six or more alternately aligned oligonucleotides, corresponding to a double-stranded DNA fragment having a desired nucleotide sequence, with equal concentrations and performing PCR under conditions involving annealing at higher temperature or those with annealing omitted (step 1), followed by a step of adding a primer set capable of amplifying the double-stranded DNA fragment of full length and further performing PCR (step 2), thus completing the present invention.

Specifically, the present invention relates to the followings.

[1] A method for producing a double-stranded DNA fragment having a desired nucleotide sequence through dual asymmetric PCR (DA-PCR), comprising:

(1) providing a plurality of oligonucleotides (sense oligonucleotides) each corresponding to a part of a sense strand of the double-stranded DNA fragment and a plurality of oligonucleotides (antisense oligonucleotides) each corresponding to a part of an antisense strand of the double-stranded DNA fragment and mixing together the oligonucleotides with equal concentrations, DNA polymerase, and dNTP to prepare a reaction mixture solution;

(2) performing PCR by using the reaction mixture solution from step (1);

(3) adding a primer set capable of amplifying the double-stranded DNA fragment of full length to the reaction mixture solution from step (2); and

(4) performing PCR by using the reaction mixture solution from step (3), wherein

when the plurality of sense oligonucleotides and the plurality of antisense oligonucleotides are aligned to the sense strand and antisense strand of the double-stranded DNA fragment, adjacent members of the sense oligonucleotides or adjacent members of the antisense oligonucleotides are not continuous with each other, the sense and antisense oligonucleotides alternately aligned each have a region having a complementary nucleotide sequence in a neighboring end part (overlap region), and the whole sequence of the double-stranded DNA fragment is covered by the sense oligonucleotides and the antisense oligonucleotides alternately aligned.

[2] The method according to [1], wherein, in step (2), a PCR profile of 94 to 98° C. for 20 to 60 seconds and 70 to 75° C. for 20 to 60 seconds is repeated in 2 to 20 cycles in the PCR.
[3] The method according to [1], wherein, in step (2), a PCR profile of 94 to 98° C. for 20 to 60 seconds, 50 to 65° C. for 5 to 60 seconds, and 70 to 75° C. for 20 to 60 seconds is repeated in 2 to 20 cycles in the PCR.
[4] The method according to any one of [1] to [3], wherein, in step (4), a PCR profile of 94 to 98° C. for 5 to 10 seconds, 50 to 65° C. for 5 to 15 seconds, and 70 to 75° C. for 5 to 30 seconds is repeated in 2 to 30 cycles in the PCR.
[5] The method according to any one of [1] to [4], wherein the DNA polymerase is a DNA polymerase selected from the group consisting of Pfu polymerase, PrimeSTAR HS DNA Polymerase, Taq polymerase, and Phusion High-Fidelity DNA Polymerase.
[6] The method according to [1] or [2], further comprising (5) performing OE-PCR.

### Advantageous Effects of Invention

The present invention provides a method that enables amplification of a double-stranded DNA fragment having a desired nucleotide sequence by accurately linking many consecutive oligonucleotides at once through DA-PCR. Further, target DNA having a desired nucleotide sequence can be synthesized in an accurate and efficient manner by combining with an OE-PCR method.

### BRIEF DESCRIPTION OF DRAWINGS

FIG. 1 shows a diagram illustrating a two-stage dual asymmetric PCR (DA-PCR) method of the present invention.

FIG. 2 shows design of oligonucleotides to produce a desired double-stranded DNA fragment having a nucleotide sequence set forth in SEQ ID NO: 1. The whole sequence of the sense strand of the desired double-stranded DNA fragment is shown, each underline indicates the nucleotide sequence of a sense oligonucleotide, and each hatched part indicates a sequence complementary to an antisense oligonucleotide.

FIG. 3 shows the structure of pUC118 plasmid DNA having a nucleotide sequence set forth in SEQ ID NO: 54.

FIG. 4 shows design of oligonucleotides to produce a desired double-stranded DNA fragment having the nucleotide sequence set forth in SEQ ID NO: 54. The whole sequence of the sense strand of the desired double-stranded DNA fragment is shown, each underline indicates the nucleotide sequence of a sense oligonucleotide, and each hatched part indicates a sequence complementary to an antisense oligonucleotide.

FIG. 5 shows results of DA-PCR using 16 oligonucleotides having nucleotide sequences set forth in SEQ ID NOs: 2 to 17 in Example 1 and Comparative Example 1.

FIG. 6 shows results of DA-PCR in Example 2.

FIG. 7 shows results of OE-PCR in Example 2.

### DESCRIPTION OF EMBODIMENTS

A method for producing a double-stranded DNA fragment having a desired nucleotide sequence through dual asymmetric PCR (DA-PCR) of one embodiment of the present invention comprises:

(1) providing a plurality of oligonucleotides (sense oligonucleotides) each corresponding to a part of a sense strand of the double-stranded DNA fragment and a plurality of oligonucleotides (antisense oligonucleotides) each corresponding to a part of an antisense strand of the double-stranded DNA fragment and mixing together the oligonucleotides with equal concentrations, DNA polymerase, and dNTP to prepare a reaction mixture solution;

(2) performing PCR by using the reaction mixture solution from step (1);

(3) adding a primer set capable of amplifying the double-stranded DNA fragment of full length to the reaction mixture solution from step (2); and

(4) performing PCR by using the reaction mixture solution from step (3).

Here, the term “double-stranded DNA fragment” is a fragment of DNA of interest, and if the DNA of interest is a double-stranded DNA encoding protein, the term refers to a double-stranded DNA fragment corresponding to a part of the DNA, and if the DNA of interest is a double-stranded cyclic DNA such as a plasmid, the term refers to a double-stranded DNA fragment corresponding to a part of the DNA. If the DNA of interest has such a short sequence that can be synthesized by using the improved DA-PCR method of the present invention, the DNA itself is included in double-stranded DNA fragments.

Step (1)

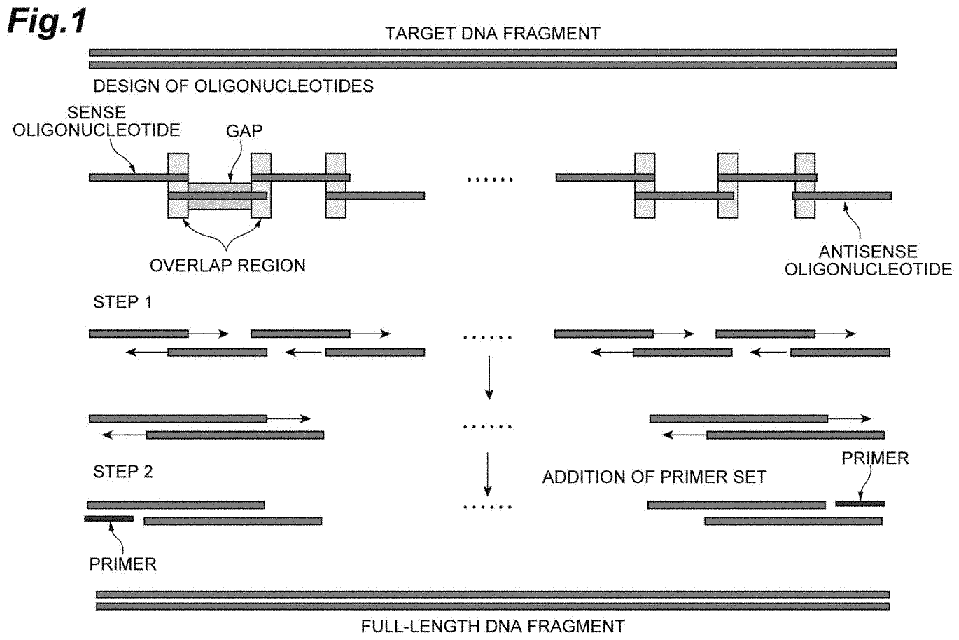

In step (1), oligonucleotides corresponding to a double-stranded DNA fragment having a desired nucleotide sequence are first provided. The oligonucleotides consist of a plurality of sense oligonucleotides each corresponding to a part of the sense strand of the double-stranded DNA fragment and a plurality of antisense oligonucleotides each corresponding to a part of the antisense strand of the double-stranded DNA fragment, and when the sense oligonucleotides and the antisense oligonucleotides are aligned to the sense strand and antisense strand of the double-stranded DNA fragment, adjacent members of the sense oligonucleotides or adjacent members of the antisense oligonucleotides are not continuous with each other, the sense and antisense oligonucleotides alternately aligned each have a region having a complementary nucleotide sequence in a neighboring end part (overlap region) (FIG. 1). Among the sense oligonucleotides and antisense oligonucleotides, oligonucleotides corresponding to an end of the sense strand or antisense strand of the double-stranded DNA fragment each have an overlap region at one end, and oligonucleotides other than the oligonucleotides corresponding to an end of the sense strand or antisense strand of the double-stranded DNA fragment each have an overlap region at both ends. In addition, each oligonucleotide has a portion other than one or two overlap regions (hereinafter, also referred to as “gap”).

In designing oligonucleotides, sense oligonucleotides or antisense oligonucleotides corresponding to the divided fragments are designed by dividing a double-stranded DNA fragment having a desired nucleotide sequence into fragments of 25 to 90 bp, preferably of 50 to 65 bp in length having one or two overlap regions and a gap, where the sense oligonucleotides or antisense oligonucleotides correspond to the divided fragments. The number of oligonucleotides can vary with the length of a target double-stranded DNA fragment and the properties of the sequence, and may be 6 to 50, 6 to 40, 6 to 32, or 6 to 30.

Each sense oligonucleotide or antisense oligonucleotide may have a length of 25 to 90 nt or 50 to 65 nt, and it is suitable that the overlap region at one end have a length of 5 to 22 nt, 10 to 22 nt, 15 to 22 nt, or 18 to 22 nt. The gap may have a length of 10 to 50 nt, 20 to 50 nt, 30 to 50 nt, or 28 to 47 nt.

The sense oligonucleotides and antisense oligonucleotides alternately aligned cover the whole sequence of a double-stranded DNA fragment (FIG. 1). Here, the phrase “cover the whole sequence” refers to the situation that oligonucleotides do not completely cover the whole nucleotide sequence with respect to a sense strand alone or an antisense strand alone, but regions not covered by sense oligonucleotides corresponding to the sense strand are covered by antisense oligonucleotides corresponding to the antisense strand, and vice versa, and hence the whole sequence is completely covered by oligonucleotides to serve as templates.

The thus-designed oligonucleotides can be synthesized to prepare by using a common, known method. Alternatively, preparation may be outsourced to a contractor for synthesis.

In step (1), a plurality of oligonucleotides with equal concentrations, DNA polymerase, and dNTP are mixed together to prepare a reaction mixture solution. The term “equal concentrations” refers to the situation that each oligonucleotide is added with roughly the same molar concentration, and the molar concentrations may be completely the same, and the concentrations may be different within a concentration range of 0.8 to 1.2 times the reference molar concentration, or different within a concentration range of 0.9 to 1.1 times the reference molar concentration. The reference molar concentration for each oligonucleotide may be, for example, 1 to 500 pmol/μl, 10 to 250 pmol/μl, or 50 to 200 pmol/μl.

The DNA polymerase is not limited to a particular DNA polymerase and may be any DNA polymerase applicable to PCR, and may be a DNA polymerase selected from the group consisting of Pfu polymerase, PrimeSTAR HS DNA Polymerase, Taq polymerase, and Phusion High-Fidelity DNA Polymerase.

For the reaction mixture solution, for example, pfu buffer (20 mM Tris-HCl, pH 9.0, 10 mM KCl, 1 mM magnesium sulfate, 6 mM (NH₄)₂SO₄, 0.1% Triton X-100, 0.1 mg/ml BSA) containing Pfu polymerase (Promega Corporation) and dNTP, as described in Non Patent Literature 3, may be used in addition to the oligonucleotides, or the oligonucleotides may be added to a commercially available reaction solution such as PrimeSTAR® Max Premix (Takara Bio Inc.) for use. Alternatively, for example, a product obtained by adding the oligonucleotides to a reaction solution containing PrimeSTAR® HS DNA Polymerase, 2 mM Mg2+, and 0.4 mM each dNTP in PrimeSTAR® Max Premix, as described in Non Patent Literature 3, may be used.

Specific examples of such reaction mixture solutions include a reaction mixture solution obtained by adding 1 μl of the mixed oligonucleotides and 24 μl of ultrapure water to 25 μl of the reaction solution PrimeSTAR® Max Premix (total volume: 50 μl).

Step (2)

In the present invention, the PCR in step (2) and the PCR in step (4) are occasionally referred to as two-stage dual asymmetric PCR (DA-PCR), collectively, and in this case the former is occasionally referred to as step 1 and the latter as step 2. In step (2), or, in step 1, polymerase chain reaction (PCR) is performed by using the reaction mixture solution from (1). The PCR in step 1 is performed under conditions shown below. Conditions on which description is not made herein can be performed according to the method described in Non Patent Literature 3.

Common PCR conditions can be used as the reaction conditions, and examples thereof include conditions involving annealing with higher-temperature setting or those with annealing omitted.

Examples of conditions with annealing omitted include those such that a PCR profile of 94 to 98° C. for 20 to 60 seconds and 70 to 75° C. for 20 to 60 seconds is repeated. The PCR profile may be repeated in 2 to 20 cycles, 5 to 20 cycles, 10 to 20, or 15 to 20 cycles.

Examples of conditions without omitting annealing include those such that a PCR profile of 94 to 98° C. for 20 to 60 seconds, 50 to 65° C. for 5 to 60 seconds, and 70 to 75° C. for 20 to 60 seconds is repeated. The PCR profile may be repeated in 2 to 20 cycles, 5 to 20 cycles, 10 to 20 cycles, or 15 to 20 cycles.

After the reaction of step 1 is performed, the reaction mixture solution in step 1 is directly used without purification for step (3), and thereafter the PCR of step 2 is performed.

Step (3)

Step (3) includes adding a primer set capable of amplifying the double-stranded DNA fragment of full length to the reaction mixture solution from (2).

As described above, the reaction mixture solution from (2) contains a mixture of linked oligonucleotides, obtained in step (2), each having increased length. To this reaction mixture solution, a primer set, specifically, a primer set of a forward primer (primer F) and a reverse primer (primer R) is added to prepare a reaction mixture solution of step (3), wherein the primer set corresponds to the nucleotide sequences at both ends expected when the mixture of linked oligonucleotides is linked to form the double-stranded DNA fragment of full length having a desired nucleotide sequence, and the primer set is capable of amplifying the double-stranded DNA fragment having a desired nucleotide sequence from the mixture of linked oligonucleotides through PCR.

Specific examples of the reaction mixture solution, for example, in using PrimeSTAR® Max Premix for the reaction mixture solution of step (1), include a reaction mixture solution obtained by adding 1 of 10 pmol/μl primer F and 1 μl of 10 pmol/μl primer R, and, as necessary, 25 μl of PrimeSTAR® Max Premix and 13 μl of distilled water to 10 μl of the reaction mixture solution from step 1 (total volume: 50 μl).

Step (4)

For reaction conditions for the PCR in step 2, a PCR profile of 94 to 98° C. for 5 to 10 seconds, 50 to 65° C. for 5 to 15 seconds, and 70 to 75° C. for 5 to 30 seconds may be repeated in 2 to 30 cycles, 5 to 30 cycles, 10 to 30 cycles, or 15 to 30 cycles.

Before initiation of the repeated cycles of the PCR profile, treatment may be performed at 94 to 98° C. for 30 to 60 seconds. After the repeated cycles of the PCR profile, treatment may be performed at 70 to 75° C. for 60 to 120 seconds. Further, treatment may be performed at 94 to 98° C. for 30 to 60 seconds before initiation of the repeated cycles of the PCR profile, and at 70 to 75° C. for 60 to 120 seconds after the repeated cycles of the PCR profile.

If the nucleotide sequences of desired DNA fragments completely cover the nucleotide sequence of the target DNA through the mixture of linked oligonucleotides, the target double-stranded DNA can be obtained.

Optional Step

(5) Overlap Extension PCR (OE-PCR)

The reaction product obtained through the PCR of step (4) may contain not only the desired double-stranded DNA fragment, but also partial fragments of the desired double-stranded DNA fragment. By purifying these DNA partial fragments and performing OE-PCR, the target double-stranded DNA can be obtained. A desired double-stranded DNA fragment obtained can be further linked to another desired double-stranded DNA fragment by performing OE-PCR.

Purification of reaction products may be performed for each reaction product obtained in step (4), or purification may be performed after equal amounts of reaction products are mixed together. Purification can be performed by extracting with phenol:chloroform:isoamylalcohol (25:24:1) with a volume equal to that of a reaction product and precipitating with a triple volume of ethanol.

Commercially available purification systems may be used. Examples of such purification systems include a Wizard® SV Gel and PCR Clean-Up System (Promega Corporation).

OE-PCR can be performed by using purified DNA partial fragments according to the method described in Non Patent Literature 3. For the reaction solution to perform OE-PCR, a reaction solution containing Pfu polymerase (Promega Corporation) and dNTP in pfu buffer (20 mM Tris-HCl, pH 9.0, 10 mM KCl, 1 mM magnesium sulfate, 6 mM (NH₄)₂SO₄, 0.1% Triton X-100, 0.1 mg/ml BSA), as described in Non Patent Literature 3, can be used. Alternatively, a commercially available reaction solution such as PrimeSTAR® Max Premix (Takara Bio Inc.) may be used.

Specific examples of the reaction mixture solution in using DNA partial fragments separately purified with a Wizard® SV Gel and PCR Clean-Up System (Promega Corporation) as template DNAs include a reaction mixture solution obtained by adding 1 μl of each of purified DNA partial fragments, 1 μl of primer F (10 pmol/μl), 1 μl of primer R (10 pmol/μl), 25 μl of PrimeSTAR® Max, and ultrapure water to reach a final volume of 50 μl, wherein primer F and primer R are a primer set corresponding to the nucleotide sequences at both ends expected when the DNA partial fragments are linked together and being capable of amplifying the DNA fragments each having a desired nucleotide sequence through PCR.

For conditions for the OE-PCR, a PCR profile of 94 to 98° C. for 20 to 60 seconds, 55 to 65° C. for 5 to 60 seconds, and 70 to 75° C. for 30 to 120 seconds may be repeated in 2 to 30 cycles, 5 to 30 cycles, 10 to 30 cycles, or 15 to 30 cycles.

(6) Amplification of Full-length DNA

The target double-stranded DNA obtained through PCR of (4) or (5) can be amplified through the following common PCR.

Specifically, amplification can be achieved by performing PCR under conditions below in 50 μl of 1×pfu buffer containing a reaction product of PCR (1 μl), 200 μM dNTP, 5 M pfu polymerase, and an outermost primer set capable of amplifying target double-stranded DNA fragments.

For conditions for the PCR, a PCR profile of 94 to 98° C. for 20 to 60 seconds, 55 to 65° C. for 20 to 60 seconds, and 70 to 75° C. for 60 to 120 seconds may be repeated in 2 to 30 cycles, 5 to 30 cycles, 10 to 30 cycles, or 15 to 30 cycles.

The final PCR product can be confirmed by agarose gel electrophoresis (1%).

### EXAMPLES

Hereinafter, the present invention will be more specifically described with reference to Examples. However, the present invention is not limited to Examples below.

(1) Design and Preparation of Oligonucleotides—Part 1

Performed were design and preparation of oligonucleotides to synthesize DNA having the nucleotide sequence set forth in SEQ ID NO: 1, resulting from modification of the nucleotide sequence of fibroin-3 from Araneus diadematus (GenBank Accession No.: M47855.1 GI:1263286), a naturally-occurring fibroin. The oligonucleotides shown in the following were designed and synthesized (outsourced to Fasmac Co., Ltd.):

SEQ ID NO: 2, which corresponds to the nucleotide sequence of positions 1 to 62 from the 5′-end of SEQ ID NO: 1 (sense strand),
SEQ ID NO: 3, which has a sequence complementary to the nucleotide sequence of positions 41 to 102 from the 5′-end of SEQ ID NO: 1 (antisense strand),
SEQ ID NO: 4, which corresponds to the nucleotide sequence of positions 81 to 142 from the 5′-end of SEQ ID NO: 1 (sense strand),
SEQ ID NO: 5, which has a sequence complementary to the nucleotide sequence of positions 121 to 182 from the 5′-end of SEQ ID NO: 1 (antisense strand),
SEQ ID NO: 6, which corresponds to the nucleotide sequence of positions 161 to 222 from the 5′-end of SEQ ID NO: 1 (sense strand),
SEQ ID NO: 7, which has a sequence complementary to the nucleotide sequence of positions 204 to 265 from the 5′-end of SEQ ID NO: 1 (antisense strand),
SEQ ID NO: 8, which corresponds to the nucleotide sequence of positions 247 to 308 from the 5′-end of SEQ ID NO: 1 (sense strand),
SEQ ID NO: 9, which has a sequence complementary to the nucleotide sequence of positions 287 to 348 from the 5′-end of SEQ ID NO: 1 (antisense strand),
SEQ ID NO: 10, which corresponds to the nucleotide sequence of positions 327 to 388 from the 5′-end of SEQ ID NO: 1 (sense strand),
SEQ ID NO: 11, which has a sequence complementary to the nucleotide sequence of positions 367 to 423 from the 5′-end of SEQ ID NO: 1 (antisense strand),
SEQ ID NO: 12, which corresponds to the nucleotide sequence of positions 402 to 448 from the 5′-end of SEQ ID NO: 1 (sense strand),
SEQ ID NO: 13, which has a sequence complementary to the nucleotide sequence of positions 427 to 478 from the 5′-end of SEQ ID NO: 1 (antisense strand),
SEQ ID NO: 14, which corresponds to the nucleotide sequence of positions 459 to 520 from the 5′-end of SEQ ID NO: 1 (sense strand),
SEQ ID NO: 15, which has a sequence complementary to the nucleotide sequence of positions 499 to 560 from the 5′-end of SEQ ID NO: 1 (antisense strand),
SEQ ID NO: 16, which corresponds to the nucleotide sequence of positions 539 to 600 from the 5′-end of SEQ ID NO: 1 (sense strand),
SEQ ID NO: 17, which has a sequence complementary to the nucleotide sequence of positions 579 to 640 from the 5′-end of SEQ ID NO: 1 (antisense strand),
SEQ ID NO: 18, which corresponds to the nucleotide sequence of positions 621 to 682 from the 5′-end of SEQ ID NO: 1 (sense strand),
SEQ ID NO: 19, which has a sequence complementary to the nucleotide sequence of positions 661 to 722 from the 5′-end of SEQ ID NO: 1 (antisense strand),
SEQ ID NO: 20, which corresponds to the nucleotide sequence of positions 701 to 755 from the 5′-end of SEQ ID NO: 1 (sense strand),
SEQ ID NO: 21, which has a sequence complementary to the nucleotide sequence of positions 734 to 790 from the 5′-end of SEQ ID NO: 1 (antisense strand),
SEQ ID NO: 22, which corresponds to the nucleotide sequence of positions 769 to 815 from the 5′-end of SEQ ID NO: 1 (sense strand),
SEQ ID NO: 23, which has a sequence complementary to the nucleotide sequence of positions 794 to 848 from the 5′-end of SEQ ID NO: 1 (antisense strand),
SEQ ID NO: 24, which corresponds to the nucleotide sequence of positions 827 to 888 from the 5′-end of SEQ ID NO: 1 (sense strand),
SEQ ID NO: 25, which has a sequence complementary to the nucleotide sequence of positions 867 to 928 from the 5′-end of SEQ ID NO: 1 (antisense strand),
SEQ ID NO: 26, which corresponds to the nucleotide sequence of positions 910 to 971 from the 5′-end of SEQ ID NO: 1 (sense strand),
SEQ ID NO: 27, which has a sequence complementary to the nucleotide sequence of positions 953 to 1014 from the 5′-end of SEQ ID NO: 1 (antisense strand),
SEQ ID NO: 28, which corresponds to the nucleotide sequence of positions 993 to 1052 from the 5′-end of SEQ ID NO: 1 (sense strand),
SEQ ID NO: 29, which has a sequence complementary to the nucleotide sequence of positions 1034 to 1090 from the 5′-end of SEQ ID NO: 1 (antisense strand),
SEQ ID NO: 30, which corresponds to the nucleotide sequence of positions 1069 to 1129 from the 5′-end of SEQ ID NO: 1 (sense strand),
SEQ ID NO: 31, which has a sequence complementary to the nucleotide sequence of positions 1108 to 1154 from the 5′-end of SEQ ID NO: 1 (antisense strand),
SEQ ID NO: 32, which corresponds to the nucleotide sequence of positions 1133 to 1187 from the 5′-end of SEQ ID NO: 1 (sense strand),
SEQ ID NO: 33, which has a sequence complementary to the nucleotide sequence of positions 1168 to 1229 from the 5′-end of SEQ ID NO: 1 (antisense strand),
SEQ ID NO: 34, which corresponds to the nucleotide sequence of positions 1210 to 1271 from the 5′-end of SEQ ID NO: 1 (sense strand),
SEQ ID NO: 35, which has a sequence complementary to the nucleotide sequence of positions 1252 to 1312 from the 5′-end of SEQ ID NO: 1 (antisense strand),
SEQ ID NO: 36, which corresponds to the nucleotide sequence of positions 1294 to 1355 from the 5′-end of SEQ ID NO: 1 (sense strand),
SEQ ID NO: 37, which has a sequence complementary to the nucleotide sequence of positions 1336 to 1397 from the 5′-end of SEQ ID NO: 1 (antisense strand),
SEQ ID NO: 38, which corresponds to the nucleotide sequence of positions 1377 to 1438 from the 5′-end of SEQ ID NO: 1 (sense strand),
SEQ ID NO: 39, which has a sequence complementary to the nucleotide sequence of positions 1419 to 1465 from the 5′-end of SEQ ID NO: 1 (antisense strand),
SEQ ID NO: 40, which corresponds to the nucleotide sequence of positions 1444 to 1505 from the 5′-end of SEQ ID NO: 1 (sense strand),
SEQ ID NO: 41, which has a sequence complementary to the nucleotide sequence of positions 1484 to 1541 from the 5′-end of SEQ ID NO: 1 (antisense strand),
SEQ ID NO: 42, which corresponds to the nucleotide sequence of positions 1522 to 1581 from the 5′-end of SEQ ID NO: 1 (sense strand),
SEQ ID NO: 43, which has a sequence complementary to the nucleotide sequence of positions 1564 to 1619 from the 5′-end of SEQ ID NO: 1 (antisense strand),
SEQ ID NO: 44, which corresponds to the nucleotide sequence of positions 1600 to 1661 from the 5′-end of SEQ ID NO: 1 (sense strand),
SEQ ID NO: 45, which has a sequence complementary to the nucleotide sequence of positions 1642 to 1703 from the 5′-end of SEQ ID NO: 1 (antisense strand),
SEQ ID NO: 46, which corresponds to the nucleotide sequence of positions 1682 to 1738 from the 5′-end of SEQ ID NO: 1 (sense strand),
SEQ ID NO: 47, which has a sequence complementary to the nucleotide sequence of positions 1717 to 1771 from the 5′-end of SEQ ID NO: 1 (antisense strand),
SEQ ID NO: 48, which corresponds to the nucleotide sequence of positions 1752 to 1813 from the 5′-end of SEQ ID NO: 1 (sense strand),
SEQ ID NO: 49, which has a sequence complementary to the nucleotide sequence of positions 1792 to 1847 from the 5′-end of SEQ ID NO: 1 (antisense strand),
SEQ ID NO: 50, which corresponds to the nucleotide sequence of positions 1826 to 1887 from the 5′-end of SEQ ID NO: 1 (sense strand),
SEQ ID NO: 51, which has a sequence complementary to the nucleotide sequence of positions 1866 to 1927 from the 5′-end of SEQ ID NO: 1 (antisense strand),
SEQ ID NO: 52, which corresponds to the nucleotide sequence of positions 1910 to 1971 from the 5′-end of SEQ ID NO: 1 (sense strand), and
SEQ ID NO: 53, which has a sequence complementary to the nucleotide sequence of positions 1953 to 1983 from the 5′-end of SEQ ID NO: 1 (antisense strand).

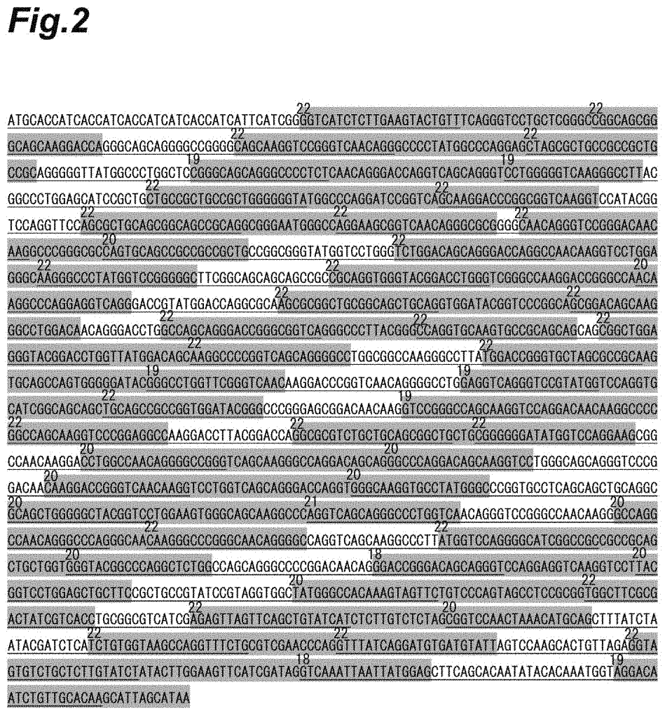

SEQ ID NOs: 2 to 53 cover the whole sequence of DNA set forth in SEQ ID NO: 1 with the configuration that sense oligonucleotides of 47 to 62 nt having sequences corresponding to the sense strand of the desired DNA fragment having the nucleotide sequence set forth in SEQ ID NO: 1 and alternately aligned antisense oligonucleotides of 31 to 62 nt having sequences corresponding to the antisense strand each have an overlap region of 18 to 22 nt having a complementary sequence, and the sense and antisense oligonucleotides are neighboring to each other in such a manner that they are hybridizing in the region, and such oligonucleotides are alternately aligned (FIG. 2). FIG. 2 shows the whole sequence of the sense strand of the desired DNA fragment, wherein each underline indicates a sense oligonucleotide, and each hatched part indicates a sequence complementary to an antisense oligonucleotide.

(2) Design and Preparation of Oligonucleotides—Part 2

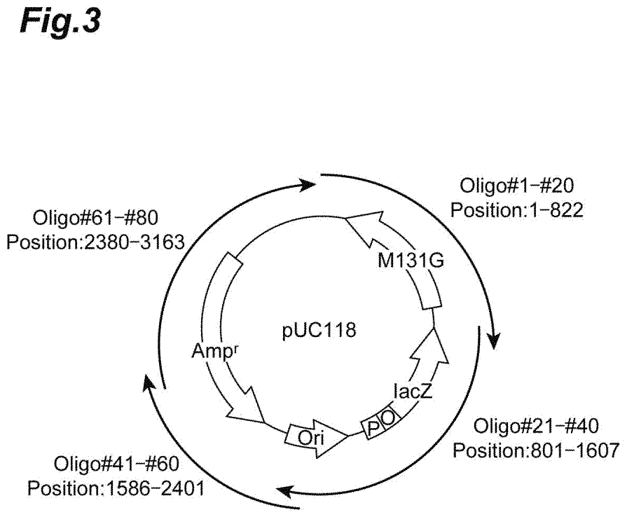

Performed in the same manner as described above were design and preparation of oligonucleotides to synthesize pUC118 plasmid DNA having a nucleotide sequence set forth in SEQ ID NO: 54 (FIG. 3). The followings were designed and synthesized (outsourced to Fasmac Co., Ltd.):

SEQ ID NO: 55, which corresponds to the nucleotide sequence of positions 1 to 60 from the 5′-end of SEQ ID NO: 54 (sense strand),
SEQ ID NO: 56, which has a sequence complementary to the nucleotide sequence of positions 41 to 100 from the 5′-end of SEQ ID NO: 54 (antisense strand),
SEQ ID NO: 57, which corresponds to the nucleotide sequence of positions 83 to 142 from the 5′-end of SEQ ID NO: 54 (sense strand),
SEQ ID NO: 58, which has a sequence complementary to the nucleotide sequence of positions 125 to 184 from the 5′-end of SEQ ID NO: 54 (antisense strand),
SEQ ID NO: 59, which corresponds to the nucleotide sequence of positions 163 to 222 from the 5′-end of SEQ ID NO: 54 (sense strand),
SEQ ID NO: 60, which has a sequence complementary to the nucleotide sequence of positions 201 to 260 from the 5′-end of SEQ ID NO: 54 (antisense strand),
SEQ ID NO: 61, which corresponds to the nucleotide sequence of positions 243 to 302 from the 5′-end of SEQ ID NO: 54 (sense strand),
SEQ ID NO: 62, which has a sequence complementary to the nucleotide sequence of positions 285 to 344 from the 5′-end of SEQ ID NO: 54 (antisense strand),
SEQ ID NO: 63, which corresponds to the nucleotide sequence of positions 327 to 385 from the 5′-end of SEQ ID NO: 54 (sense strand),
SEQ ID NO: 64, which has a sequence complementary to the nucleotide sequence of positions 368 to 427 from the 5′-end of SEQ ID NO: 54 (antisense strand),
SEQ ID NO: 65, which corresponds to the nucleotide sequence of positions 407 to 466 from the 5′-end of SEQ ID NO: 54 (sense strand),
SEQ ID NO: 66, which has a sequence complementary to the nucleotide sequence of positions 449 to 508 from the 5′-end of SEQ ID NO: 54 (antisense strand),
SEQ ID NO: 67, which corresponds to the nucleotide sequence of positions 490 to 546 from the 5′-end of SEQ ID NO: 54 (sense strand),
SEQ ID NO: 68, which has a sequence complementary to the nucleotide sequence of positions 529 to 588 from the 5′-end of SEQ ID NO: 54 (antisense strand),
SEQ ID NO: 69, which corresponds to the nucleotide sequence of positions 571 to 630 from the 5′-end of SEQ ID NO: 54 (sense strand),
SEQ ID NO: 70, which has a sequence complementary to the nucleotide sequence of positions 612 to 671 from the 5′-end of SEQ ID NO: 54 (antisense strand),
SEQ ID NO: 71, which corresponds to the nucleotide sequence of positions 651 to 708 from the 5′-end of SEQ ID NO: 54 (sense strand),
SEQ ID NO: 72, which has a sequence complementary to the nucleotide sequence of positions 687 to 746 from the 5′-end of SEQ ID NO: 54 (antisense strand),
SEQ ID NO: 73, which corresponds to the nucleotide sequence of positions 725 to 784 from the 5′-end of SEQ ID NO: 54 (sense strand),
SEQ ID NO: 74, which has a sequence complementary to the nucleotide sequence of positions 765 to 822 from the 5′-end of SEQ ID NO: 54 (antisense strand),
SEQ ID NO: 75, which corresponds to the nucleotide sequence of positions 801 to 860 from the 5′-end of SEQ ID NO: 54 (sense strand),
SEQ ID NO: 76, which has a sequence complementary to the nucleotide sequence of positions 839 to 898 from the 5′-end of SEQ ID NO: 54 (antisense strand),
SEQ ID NO: 77, which corresponds to the nucleotide sequence of positions 877 to 936 from the 5′-end of SEQ ID NO: 54 (sense strand),
SEQ ID NO: 78, which has a sequence complementary to the nucleotide sequence of positions 915 to 974 from the 5′-end of SEQ ID NO: 54 (antisense strand),
SEQ ID NO: 79, which corresponds to the nucleotide sequence of positions 953 to 1010 from the 5′-end of SEQ ID NO: 54 (sense strand),
SEQ ID NO: 80, which has a sequence complementary to the nucleotide sequence of positions 989 to 1046 from the 5′-end of SEQ ID NO: 54 (antisense strand),
SEQ ID NO: 81, which corresponds to the nucleotide sequence of positions 1029 to 1088 from the 5′-end of SEQ ID NO: 54 (sense strand),
SEQ ID NO: 82, which has a sequence complementary to the nucleotide sequence of positions 1071 to 1130 from the 5′-end of SEQ ID NO: 54 (antisense strand),
SEQ ID NO: 83, which corresponds to the nucleotide sequence of positions 1113 to 1171 from the 5′-end of SEQ ID NO: 54 (sense strand),
SEQ ID NO: 84, which has a sequence complementary to the nucleotide sequence of positions 1154 to 1213 from the 5′-end of SEQ ID NO: 54 (antisense strand),
SEQ ID NO: 85, which corresponds to the nucleotide sequence of positions 1192 to 1251 from the 5′-end of SEQ ID NO: 54 (sense strand),
SEQ ID NO: 86, which has a sequence complementary to the nucleotide sequence of positions 1234 to 1293 from the 5′-end of SEQ ID NO: 54 (antisense strand),
SEQ ID NO: 87, which corresponds to the nucleotide sequence of positions 1272 to 1331 from the 5′-end of SEQ ID NO: 54 (sense strand),
SEQ ID NO: 88, which has a sequence complementary to the nucleotide sequence of positions 1310 to 1369 from the 5′-end of SEQ ID NO: 54 (antisense strand),
SEQ ID NO: 89, which corresponds to the nucleotide sequence of positions 1348 to 1406 from the 5-end of SEQ ID NO: 54 (sense strand),
SEQ ID NO: 90, which has a sequence complementary to the nucleotide sequence of positions 1387 to 1446 from the 5′-end of SEQ ID NO: 54 (antisense strand),
SEQ ID NO: 91, which corresponds to the nucleotide sequence of positions 1429 to 1488 from the 5′-end of SEQ ID NO: 54 (sense strand),
SEQ ID NO: 92, which has a sequence complementary to the nucleotide sequence of positions 1471 to 1530 from the 5′-end of SEQ ID NO: 54 (antisense strand),
SEQ ID NO: 93, which corresponds to the nucleotide sequence of positions 1510 to 1569 from the 5′-end of SEQ ID NO: 54 (sense strand),
SEQ ID NO: 94, which has a sequence complementary to the nucleotide sequence of positions 1548 to 1607 from the 5′-end of SEQ ID NO: 54 (antisense strand),
SEQ ID NO: 95, which corresponds to the nucleotide sequence of positions 1586 to 1645 from the 5′-end of SEQ ID NO: 54 (sense strand),
SEQ ID NO: 96, which has a sequence complementary to the nucleotide sequence of positions 1628 to 1687 from the 5′-end of SEQ ID NO: 54 (antisense strand),
SEQ ID NO: 97, which corresponds to the nucleotide sequence of positions 1666 to 1725 from the 5′-end of SEQ ID NO: 54 (sense strand),
SEQ ID NO: 98, which has a sequence complementary to the nucleotide sequence of positions 1704 to 1763 from the 5′-end of SEQ ID NO: 54 (antisense strand),
SEQ ID NO: 99, which corresponds to the nucleotide sequence of positions 1746 to 1805 from the 5′-end of SEQ ID NO: 54 (sense strand),
SEQ ID NO: 100, which has a sequence complementary to the nucleotide sequence of positions 1788 to 1847 from the 5′-end of SEQ ID NO: 54 (antisense strand),
SEQ ID NO: 101, which corresponds to the nucleotide sequence of positions 1827 to 1885 from the 5′-end of SEQ ID NO: 54 (sense strand),
SEQ ID NO: 102, which has a sequence complementary to the nucleotide sequence of positions 1867 to 1926 from the 5′-end of SEQ ID NO: 54 (antisense strand),
SEQ ID NO: 103, which corresponds to the nucleotide sequence of positions 1909 to 1965 from the 5′-end of SEQ ID NO: 54 (sense strand),
SEQ ID NO: 104, which has a sequence complementary to the nucleotide sequence of positions 1944 to 2003 from the 5′-end of SEQ ID NO: 54 (antisense strand),
SEQ ID NO: 105, which corresponds to the nucleotide sequence of positions 1984 to 2043 from the 5′-end of SEQ ID NO: 54 (sense strand),
SEQ ID NO: 106, which has a sequence complementary to the nucleotide sequence of positions 2022 to 2081 from the 5-end of SEQ ID NO: 54 (antisense strand),
SEQ ID NO: 107, which corresponds to the nucleotide sequence of positions 2063 to 2122 from the 5′-end of SEQ ID NO: 54 (sense strand),
SEQ ID NO: 108, which has a sequence complementary to the nucleotide sequence of positions 2105 to 2164 from the 5′-end of SEQ ID NO: 54 (antisense strand),
SEQ ID NO: 109, which corresponds to the nucleotide sequence of positions 2145 to 2204 from the 5′-end of SEQ ID NO: 54 (sense strand),
SEQ ID NO: 110, which has a sequence complementary to the nucleotide sequence of positions 2186 to 2245 from the 5′-end of SEQ ID NO: 54 (antisense strand),
SEQ ID NO: 111, which corresponds to the nucleotide sequence of positions 2224 to 2283 from the 5′-end of SEQ ID NO: 54 (sense strand),
SEQ ID NO: 112, which has a sequence complementary to the nucleotide sequence of positions 2262 to 2321 from the 5′-end of SEQ ID NO: 54 (antisense strand),
SEQ ID NO: 113, which corresponds to the nucleotide sequence of positions 2301 to 2360 from the 5′-end of SEQ ID NO: 54 (sense strand),
SEQ ID NO: 114, which has a sequence complementary to the nucleotide sequence of positions 2343 to 2401 from the 5′-end of SEQ ID NO: 54 (antisense strand),
SEQ ID NO: 115, which corresponds to the nucleotide sequence of positions 2380 to 2439 from the 5′-end of SEQ ID NO: 54 (sense strand),
SEQ ID NO: 116, which has a sequence complementary to the nucleotide sequence of positions 2421 to 2480 from the 5′-end of SEQ ID NO: 54 (antisense strand),
SEQ ID NO: 117, which corresponds to the nucleotide sequence of positions 2463 to 2522 from the 5′-end of SEQ ID NO: 54 (sense strand),
SEQ ID NO: 118, which has a sequence complementary to the nucleotide sequence of positions 2505 to 2562 from the 5′-end of SEQ ID NO: 54 (antisense strand),
SEQ ID NO: 119, which corresponds to the nucleotide sequence of positions 2541 to 2600 from the 5′-end of SEQ ID NO: 54 (sense strand),
SEQ ID NO: 120, which has a sequence complementary to the nucleotide sequence of positions 2579 to 2638 from the 5′-end of SEQ ID NO: 54 (antisense strand),
SEQ ID NO: 121, which corresponds to the nucleotide sequence of positions 2620 to 2679 from the 5′-end of SEQ ID NO: 54 (sense strand),
SEQ ID NO: 122, which has a sequence complementary to the nucleotide sequence of positions 2661 to 2720 from the 5′-end of SEQ ID NO: 54 (antisense strand),
SEQ ID NO: 123, which corresponds to the nucleotide sequence of positions 2700 to 2759 from the 5′-end of SEQ ID NO: 54 (sense strand),
SEQ ID NO: 124, which has a sequence complementary to the nucleotide sequence of positions 2741 to 2800 from the 5′-end of SEQ ID NO: 54 (antisense strand),
SEQ ID NO: 125, which corresponds to the nucleotide sequence of positions 2781 to 2840 from the 5′-end of SEQ ID NO: 54 (sense strand),
SEQ ID NO: 126, which has a sequence complementary to the nucleotide sequence of positions 2822 to 2881 from the 5′-end of SEQ ID NO: 54 (antisense strand),
SEQ ID NO: 127, which corresponds to the nucleotide sequence of positions 2864 to 2923 from the 5′-end of SEQ ID NO: 54 (sense strand),
SEQ ID NO: 128, which has a sequence complementary to the nucleotide sequence of positions 2906 to 2963 from the 5′-end of SEQ ID NO: 54 (antisense strand),
SEQ ID NO: 129, which corresponds to the nucleotide sequence of positions 2946 to 3005 from the 5′-end of SEQ ID NO: 54 (sense strand),
SEQ ID NO: 130, which has a sequence complementary to the nucleotide sequence of positions 2988 to 3047 from the 5′-end of SEQ ID NO: 54 (antisense strand),
SEQ ID NO: 131, which corresponds to the nucleotide sequence of positions 3027 to 3086 from the 5′-end of SEQ ID NO: 54 (sense strand),
SEQ ID NO: 132, which has a sequence complementary to the nucleotide sequence of positions 3065 to 3122 from the 5′-end of SEQ ID NO: 54 (antisense strand),
SEQ ID NO: 133, which corresponds to the nucleotide sequence of positions 3101 to 3160 from the 5′-end of SEQ ID NO: 54 (sense strand), and
SEQ ID NO: 134, which has a sequence complementary to the nucleotide sequence of positions 3143 to 3163 from the 5′-end of SEQ ID NO: 54 (antisense strand).

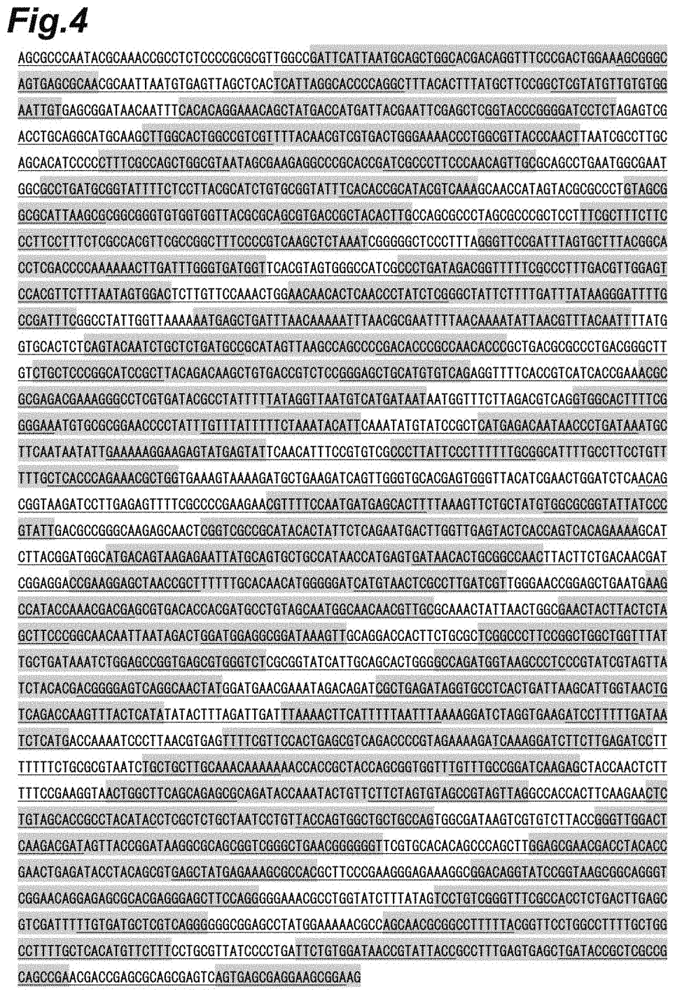

The whole sequence of DNA set forth in SEQ ID NO: 54 is covered with the configuration that sense oligonucleotides of 57 to 60 nt having sequences corresponding to the sense strand of the desired DNA set forth in SEQ ID NO: 54 and alternately aligned antisense oligonucleotides of 21 to 60 nt having sequences corresponding to the antisense strand each have an overlap region of 18 to 22 nt having a complementary sequence, and the sense and antisense oligonucleotides are neighboring to each other in such a manner that they are hybridizing in the region, and such oligonucleotides are alternately aligned (FIG. 4). FIG. 4 shows the whole sequence of the sense strand of the desired DNA fragment, wherein each underline indicates a sense oligonucleotide, and each hatched part indicates a sequence complementary to an antisense oligonucleotide.

### Comparative Example 1

In the conventional DA-PCR method described in Non Patent Literature 3, each four continuous oligonucleotides are mixed together in such a manner that the amount of moles of each of the two outer oligonucleotides is excessive and five times that of each of the inner oligonucleotides, and one-stage DA-PCR is performed under conditions at 94° C. for 20 seconds, at 45° C. for 15 seconds, and at 72° C. for 30 seconds.

In Comparative Example 1, DA-PCR according to the conventional method was performed with 16 oligonucleotides having the nucleotide sequences set forth in SEQ ID NOs: 2 to 17.

Specifically, a reaction mixture solution was prepared by adding 25 μl of PrimeSTAR® Max to 7.6 μl of a solution obtained by mixing the 16 oligonucleotides in such a manner that the amount of moles (10 pmol) of each of the two outer oligonucleotides (SEQ ID NO: 2 and SEQ ID NO: 17) was excessive and five times that (2 pmol) of each of the inner oligonucleotides, and adding ultrapure water thereto to reach a final volume of 50 μl.

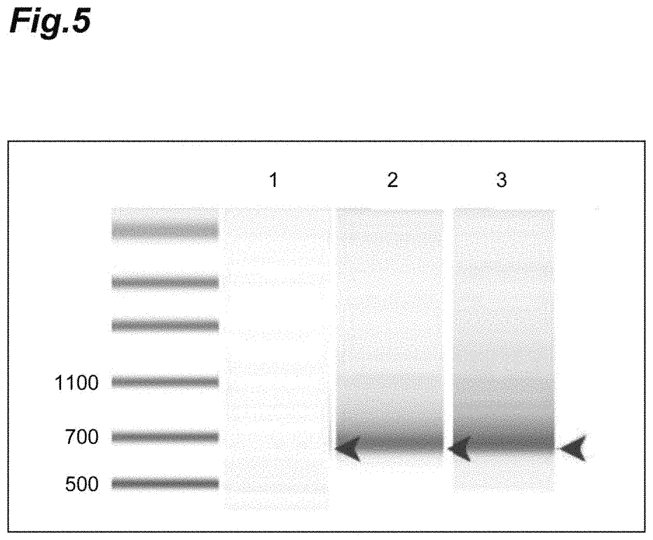

If the conventional method is used for the reaction mixture solution, a PCR profile of 94° C., 45° C., and 72° C. is to be repeated; however, it is clear that linkage is not successfully caused under such conditions, and hence PCR was performed under conditions with higher denaturation temperature and annealing temperature of 98° C., 65° C., and 72° C., which are close to the temperatures in the present invention. Specifically, heating was performed at 98° C. for 5 minutes, and thereafter a PCR profile of 98° C. for 10 seconds, 65° C. for 5 seconds, and 72° C. for 10 seconds was repeated in 20 cycles, and further the resultant was reacted at 72° C. for 2 minutes. The result is shown in lane 1 in FIG. 5. Even with the elevated annealing temperature, the conventional method failed in linking the 16 fragments.

### Example 1

To 1 μl of a solution obtained by mixing equal amounts of moles (100 pmol) of the 16 oligonucleotides having the nucleotide sequences set forth in SEQ ID NOs: 2 to 17, 25 μl of PrimeSTAR® Max was added, to which ultrapure water was added to reach a final volume of 50 μl to prepare a reaction mixture solution.

As step 1, a PCR profile of 98° C. for 10 seconds, 65° C. for 5 seconds, and 72° C. for 5 seconds was repeated in 15 cycles.

To 10 μl of the reaction solution obtained in step 1, 1 μl of oligo DNA (10 pmol/μl) set forth in SEQ ID NO: 2 as primer F, 1 μl of oligo DNA (10 pmol/μl) set forth in SEQ ID NO: 17 as primer R, and 25 μl of PrimeSTAR® Max were added, and ultrapure water was added thereto to reach a final volume of 50 μl to prepare a reaction mixture solution, which was heated, as step 2, at 98° C. for 1 minute and thereafter a PCR profile of 98° C. for 10 seconds, 65° C. for 5 seconds, and 72° C. for 15 seconds was repeated in 20 cycles, and the resultant was further reacted at 72° C. for 2 minutes.

The result is shown in lane 2 in FIG. 5. A band of DNA was successfully detected at a target position. Even when the annealing at 65° C. was not performed in step 1 and a PCR profile of 98° C. for 60 seconds and 72° C. for 60 seconds was repeated in 15 cycles, a band was successfully detected at a target position, similarly (lane 3 in FIG. 5).

### Example 2

To 1 μl of a solution obtained by mixing equal amounts of moles (100 pmol) of 20 oligonucleotides having the nucleotide sequences set forth in SEQ ID NOs: 55 to 74 (#1-20), 25 μl of PrimeSTAR® Max was added, and ultrapure water was added thereto to reach a final volume of 50 μl to prepare a reaction mixture solution. In the same manner, 25 μl of PrimeSTAR® Max was added to 1 μl of each of a solution obtained by mixing equal amounts of moles (100 pmol) of 20 oligonucleotides having the nucleotide sequences set forth in SEQ ID NOs: 75 to 94 (#21-40), a solution obtained by mixing equal amounts of moles (100 pmol) of 20 oligonucleotides having the nucleotide sequences set forth in SEQ ID NOs: 95 to 114 (#41-60), a solution obtained by mixing equal amounts of moles (100 pmol) of 20 oligonucleotides having the nucleotide sequences set forth in SEQ ID NOs: 115 to 134 (#61-80), a solution obtained by mixing equal amounts of moles (100 pmol) of 40 oligonucleotides having the nucleotide sequences set forth in SEQ ID NOs: 55 to 94 (#1-40), and a solution obtained by mixing equal amounts of moles (100 pmol) of 40 oligonucleotides having the nucleotide sequences set forth in SEQ ID NOs: 95 to 134 (#41-80), and ultrapure water was added thereto to reach a final volume of 50 μl to prepare six reaction mixture solutions in total.

As step 1, a PCR profile of 98° C. for 10 seconds, 50 to 65° C. for 5 seconds, and 72° C. for 10 seconds was repeated in 15 cycles with each of the thus-prepared reaction mixture solutions.

To 10 μl of each reaction solution obtained in step 1, 1 μl of primer F (10 pmol/μl) and 1 μl of primer R (10 pmol/μl) and 25 μl of PrimeSTAR® Max were prepared, and ultrapure water was prepared thereto to reach a final volume of 50 μl, wherein primer F and primer R are a primer set corresponding to the nucleotide sequences at both ends expected when the oligonucleotides were linked together in step 1 and being capable of amplifying the mixture of linked oligonucleotides through PCR, and each of the resultants was heated, as step 2, at 98° C. for 1 minute and thereafter a PCR profile of 98° C. for 10 seconds, 50 to 65° C. for 5 seconds, and 72° C. for 30 seconds was repeated in 20 cycles, and each of the resultants was further reacted at 72° C. for 2 minutes. The results were confirmed through capillary electrophoresis using a LabChip GX system (PerkinElmer).

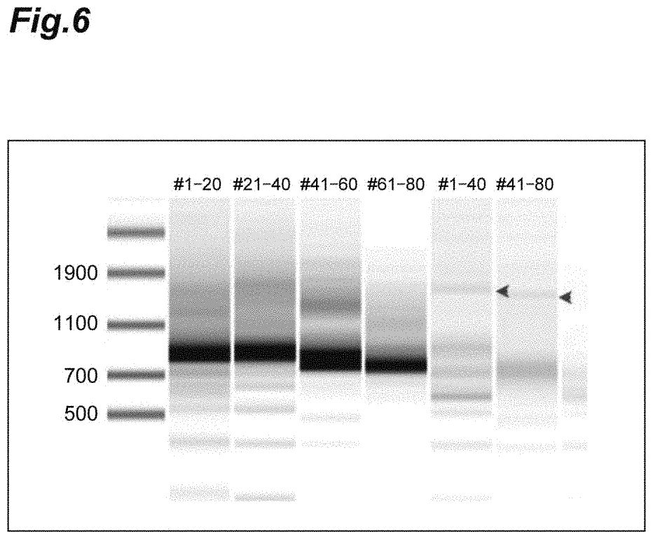

When the annealing temperature in step 1 was, for example, 50° C., the annealing temperature in step 2 was also set to 50° C., that is, the annealing temperatures were correspondingly set. Annealing was performed under different conditions within a range of 50 to 65° C., and the results when annealing was performed at 50° C. are shown in FIG. 6. For each of the oligonucleotide mixture reaction solutions of 20 fragments, a band was successfully detected clearly at a target position (lanes #1-20, #21-40, #41-60, and #61-80). For each of the cases with 40 fragments, similarly, a band was successfully detected at a target position, though the band was pale (lanes #1-40, #41-80). The DNA fragments obtained were purified in the following manner, and OE-PCR was performed.

PCR-amplified DNA fragments #1-20, #21-40, #41-60, #61-80, #1-40, and #41-80 obtained above were purified by treating with a Wizard® SV Gel and PCR Clean-Mp System (produced by Promega Corporation, product No.: A9280), a commercially available purification system, in accordance with an accompanying catalog.

Equal amounts of DNA fragments #1-20 and #21-40 purified were mixed together to prepare template DNA 1. In the same manner, template DNA 2 was prepared from DNA fragments #41-60 and #61-80, and template DNA 3 from DNA fragments #1-40 and #41-80.

For template DNA 1 to link DNA fragments #1-20 and #21-40 together, the oligonucleotide set forth in SEQ ID NO: 55 as primer F and the oligonucleotide set forth in SEQ ID NO: 94 as primer R were used.

For template DNA 2 to link DNA fragments #41-60 and #61-80 together, the oligonucleotide set forth in SEQ ID NO: 95 as primer F and the oligonucleotide set forth in SEQ ID NO: 134 as primer R were used.

For template DNA 3 to link DNA fragments #1-40 and #41-80 together, the oligonucleotide set forth in SEQ ID NO: 55 as primer F and the oligonucleotide set forth in SEQ ID NO: 134 as primer R were used.

Reaction mixture solutions were prepared by adding 1 μl of primer F and 1 μl of primer R each adjusted to 10 pmol/μl, and 25 μl of PrimeSTAR® Max to 2 μl of template DNA 1 or template DNA 2 or 4 μl of template DNA 3, and adding ultrapure water thereto to reach a final volume of 50 μl.

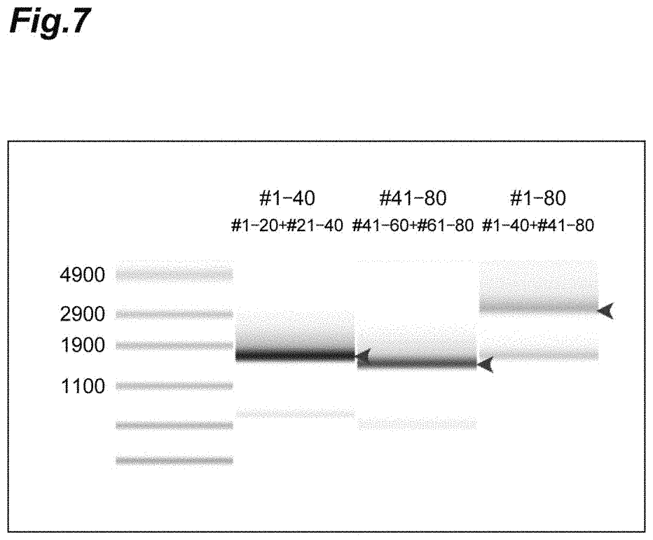

Each of the reaction mixture solutions prepared was heated at 98° C. for 60 seconds and thereafter a PCR profile of 98° C. for 10 seconds, 65° C. for 5 seconds, and 72° C. for 30 seconds was repeated in 20 cycles, and each of the resultants was further reacted at 72° C. for 2 minutes. FIG. 7 shows confirmation of the results through capillary electrophoresis using a LabChip GX system (PerkinElmer). DNA fragments had been linked as intended for any of the template DNAs.

1. A method for producing a double-stranded DNA fragment having a desired nucleotide sequence through dual asymmetric PCR (DA-PCR), comprising:
(1) providing a plurality of oligonucleotides (sense oligonucleotides) each corresponding to a part of a sense strand of the double-stranded DNA fragment and a plurality of oligonucleotides (antisense oligonucleotides) each corresponding to a part of an antisense strand of the double-stranded DNA fragment and mixing together the oligonucleotides with equal concentrations, DNA polymerase, and dNTP to prepare a reaction mixture solution;
(2) performing PCR by using the reaction mixture solution from step (1);
(3) adding a primer set capable of amplifying the double-stranded DNA fragment of full length to the reaction mixture solution from step (2); and
(4) performing PCR by using the reaction mixture solution from step (3), wherein
when the plurality of sense oligonucleotides and the plurality of antisense oligonucleotides are aligned to the sense strand and antisense strand of the double-stranded DNA fragment, adjacent members of the sense oligonucleotides or adjacent members of the antisense oligonucleotides are not continuous with each other, the sense and antisense oligonucleotides alternately aligned each have a region having a complementary nucleotide sequence in a neighboring end part (overlap region), and a whole sequence of the double-stranded DNA fragment is covered by the sense oligonucleotides and the antisense oligonucleotides alternately aligned.

2. The method according to claim 1, wherein, in step (2), a PCR profile of 94 to 98° C. for 20 to 60 seconds and 70 to 75° C. for 20 to 60 seconds is repeated in 2 to 20 cycles in the PCR.

3. The method according to claim 1, wherein, in step (2), a PCR profile of 94 to 98° C. for 20 to 60 seconds, 50 to 65° C. for 5 to 60 seconds, and 70 to 75° C. for 20 to 60 seconds is repeated in 2 to 20 cycles in the PCR.

4. The method according to claim 1, wherein, in step (4), a PCR profile of 94 to 98° C. for 5 to 10 seconds, 50 to 65° C. for 5 to 15 seconds, and 70 to 75° C. for 5 to 30 seconds is repeated in 2 to 30 cycles in the PCR.

5. The method according to claim 1, wherein the DNA polymerase is a DNA polymerase selected from the group consisting of Pfu polymerase, PrimeSTAR HS DNA Polymerase, Taq polymerase, and Phusion High-Fidelity DNA Polymerase.

6. The method according to claim 1, further comprising (5) performing OE-PCR.

7. The method according to claim 2, further comprising (5) performing OE-PCR.
